(19) United States
(12) Patent Application Publication
(10) Pub. No.: US 2025/0084451 A1
(43) Pub. Date: Mar. 13, 2025

(54) Method and Device for Detecting Presence, Concentration, and Antibiotic Resistance of Bacteria in Urine Samples

(71) Applicant: Astek Diagnostics Inc., Baltimore, MD (US)

(72) Inventors: Mustafa Al-Adhami, Elkridge, MD (US); Courtney Cavin, Pasadena, MD (US)

(21) Appl. No.: 18/765,978

(22) Filed: Jul. 8, 2024

Related U.S. Application Data
(60) Provisional application No. 63/525,510, filed on Jul. 7, 2023.

(51) Int. Cl. C12Q 1/18; B01L 3/00; G01N 21/64

(52) U.S. Cl. CPC C12Q 1/18; B01L 3/502753; G01N 21/6428; B01L 2200/028; B01L 2200/0647; B01L 2200/16; B01L 2300/0681; G01N 2021/6439

(57) ABSTRACT

An automated system that determines the presence of bacteria in a urine sample, concentration of the bacteria, and the bacteria's resistance to a panel of antibiotics, in around one hour instead of days. An automated assay to detect cell viability in mammalian cell cultures using reduction of alamarBlue through oxidation by NADH to NAD+. The assay is conducted within a fluidic chip, wherein a sample is mixed with alamarBlue, a weakly fluorescent indicator dye. Its rate of conversion to a highly fluorescent compound is monitored using a kinetics fluorimeter for 15 minutes, and metabolic activity is shown by an increase in fluorescence. A sample with live bacteria displays a consistent and rapid increase in fluorescence, whereas the negative control exhibits little to no increase over time.

### FIELD OF THE INVENTION

The invention relates to diagnostic methods and devices for detecting bacteria in urine.

### BACKGROUND OF THE INVENTION

Traditional UTI treatment consists of three steps: 1) determining if the patient has a UTI through clinical evaluation and urinalysis; 2) identification of the bacteria causing the infection, typically achieved through culturing methods, which can take days; 3) antibiotic sensitivity testing to select the most effective antibiotic for the specific bacterial strain.

### SUMMARY OF THE INVENTION

The present invention is an automated system that determines the presence of bacteria in a urine sample, concentration of the bacteria, and the bacteria's resistance to a panel of antibiotics, skipping the days-long second step, allowing healthcare professionals to directly proceed with antibiotic sensitivity testing without the need for bacterial identification. This shortens the time required to obtain antibiotic sensitivity results from days to just one hour, enabling faster and more accurate treatment decisions. The expedited process not only benefits patients by delivering quicker relief but also helps in the fight against antibiotic resistance, as it minimizes the use of broad-spectrum antibiotics and promotes targeted therapy.

The detection system of the invention automates an assay commonly used to detect cell viability in mammalian cell cultures: reduction of alamar Blue through oxidation by NADH to NAD+. The conversion rate is proportional to the bacterial metabolic rate and concentration. The assay is conducted within a fluidic chip, wherein a sample is mixed with alamarBlue, a weakly fluorescent indicator dye. Its rate of conversion to a highly fluorescent compound is monitored using a kinetics fluorimeter for 15 minutes, and metabolic activity is shown by an increase in fluorescence. A sample with live bacteria displays a consistent and rapid increase in fluorescence, whereas the negative control exhibits little to no increase over time.

The difference of the slopes (sample-control) is proportional to the live bacterial concentration in the sample. This technique was shown to be capable of detecting bacterial infections at a level of 10 CFU (Colony Forming Units)/mL with clinically-relevant bacteria.

To transform the alamarBlue assay from a process that typically takes several hours to one that only takes 15 minutes, the following optimizations and modifications are implemented:
1. Enhanced penetration: Use sodium salicylate or other agents to shrink the polysaccharide shell of bacteria, allowing for better diffusion of the alamarBlue dye and other assay components into the cells, thus accelerating the reduction of resazurin to resorufin.
2. Closed analysis chamber: The analysis chamber is closed to the atmosphere, which increases the sensitivity of the device by maintaining a stable and controlled environment for the assay.
3. Methyl Viologen: Methyl Viologen Dichloride Hydrate is introduced to the assay to increase NADH production, enhancing the reduction of resazurin to resorufin and speeding up the overall assay process.
4. Enhanced detection system: a sensitive single-excitation, single-emission kinetics fluorometer is used with a carefully selected filter to measure the fluorescence signal of resorufin accurately and efficiently, allowing for real-time monitoring of the assay progress.

### BRIEF DESCRIPTION OF DRAWINGS

FIG. 1 is a flow chart showing the steps performed according to one embodiment of the invention.

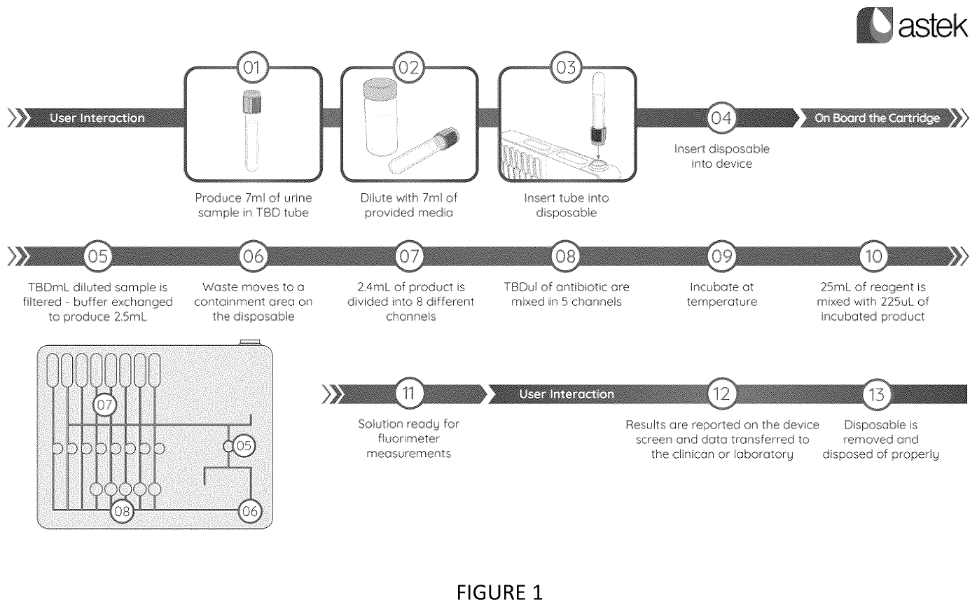

FIG. 2 is a perspective view of a multi-channel fluidic cartridge analysis device according to an embodiment of the invention.

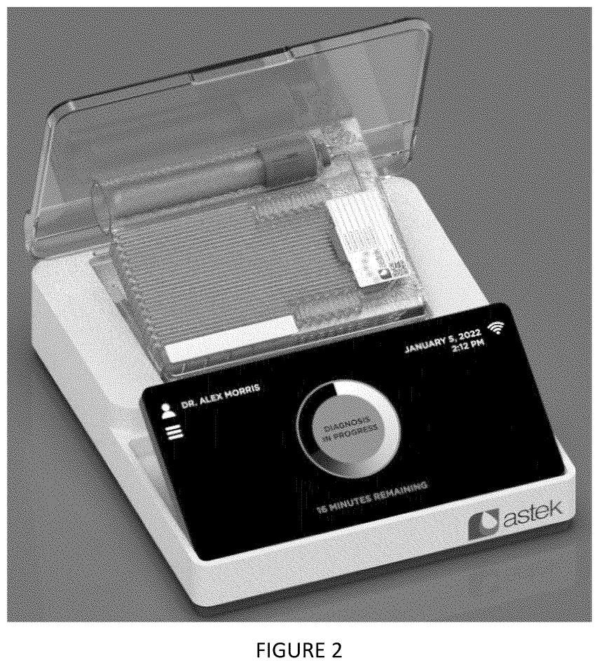

FIG. 3 is a perspective view of a multi-channel fluidic cartridge analysis device according to another embodiment of the invention.

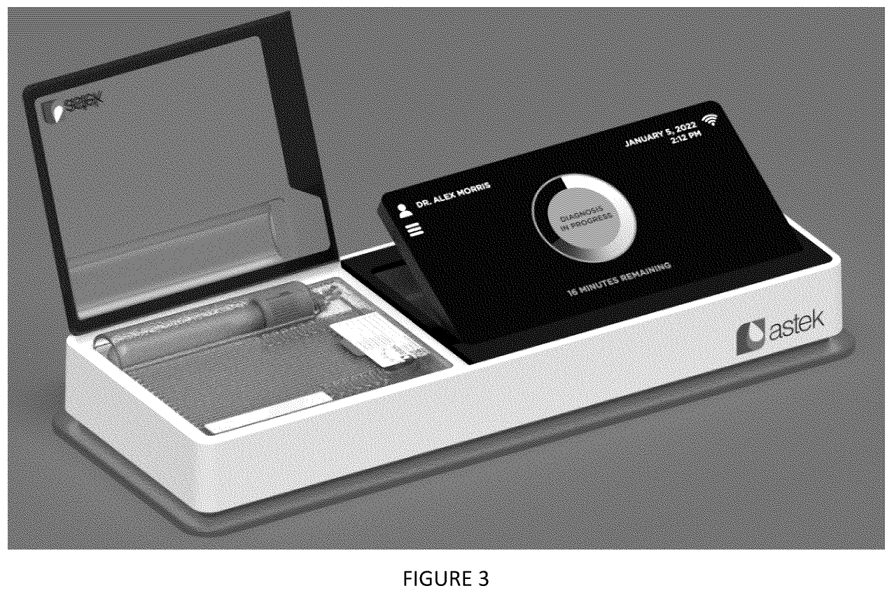

FIG. 4 is a perspective view of a disposable multi-channel fluidic cartridge according to an embodiment of the invention.

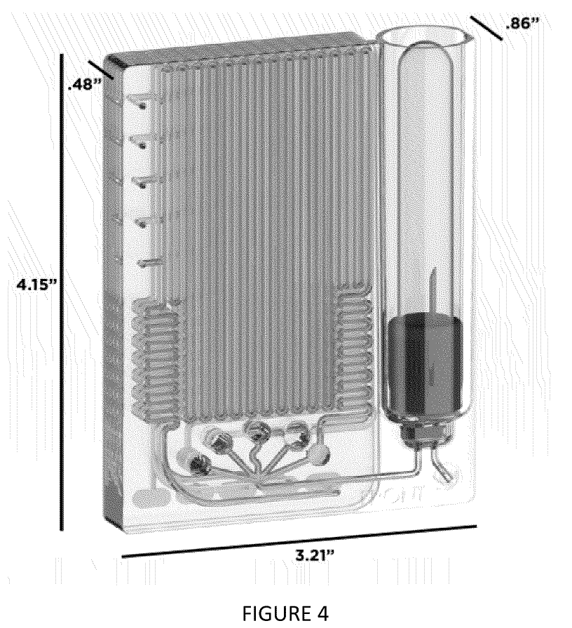

FIG. 5 is a representation of a cross-flow membrane chip according to an embodiment of the invention.

### DETAILED DESCRIPTION OF THE INVENTION

Sample Introduction: The user begins by transferring a patient's urine from a urine collection cup to a standard Vacutainer or other sterile vacuum-sealed container with a rubber cap designed to minimize contamination risks. Once the sample is collected, the user inserts the Vacutainer into the designated slot or holder on a multi-channel fluidic cartridge analysis device. The holder is specifically designed to securely accommodate the Vacutainer, ensuring proper alignment with the sample transfer needles in the device to facilitate seamless sample transfer. The multi-channel fluidic cartridge analysis device features a removable, preferably disposable, cartridge containing a plurality of separate channels, each channel preferably having a buffer exchange section and an antibiotic contact/mixing section containing an antibiotic-loaded matrix. A pressure pump and a series of valves are employed to direct the urine fluid into and through the separate channels. Any type of pump may be used, including peristaltic, syringe, diaphragm, piezoelectric, electroosmotic, and centrifugal.

Upon insertion, the system initiates the sample transfer process, during which two needles, one short and one long, pierce the rubber cap of the Vacutainer. The short needle is responsible for venting, allowing air to flow into the container and equalize the pressure as the sample is withdrawn. Meanwhile, the long needle, connected to microfluidic channels within the fluidic cartridge, aspirates the urine sample into the cartridge. Once the urine sample has been drawn into the fluidic cartridge's fluidic channels, it is divided into a plurality of different channels with a series of active or passive solenoid valves. Each channel will then go through a buffer exchange process. Current embodiments have eight channels, but the device may be manufactured/used with any number of channels.

The remainder of the process as described below, takes place in each of the different channels unless specified otherwise.

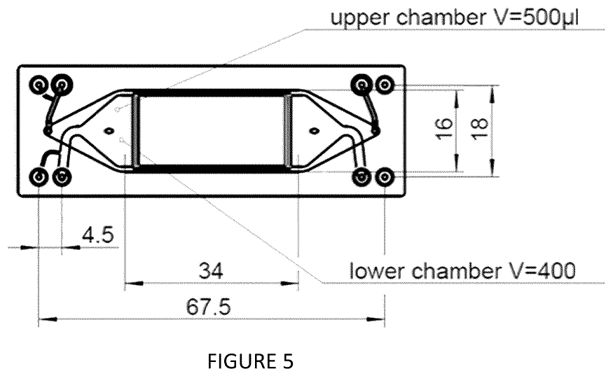

Buffer Exchange: The fluidic cartridge integrates a buffer exchange process using a filter (preferably a cross flow filter or dead end filter) to isolate bacteria from urine and suspend them in media. In the embodiment described herein, the cross-flow membrane chip (FIG. 5) features two separate chambers, each linked by a permeable membrane. Both chambers are equipped with their own inlet and outlet, allowing for independent fluid flow and exchange through the connecting membrane. In this process, the urine sample containing bacteria is passed through the filter, which separates the bacteria from the fluid. The filtrate, or permeate, consists of purified, bacteria-free fluid, while the retentate contains concentrated bacteria. This filtration method enables the reduction of the initial 1 mL bacteria-in-urine sample to a smaller, 250 μL volume of bacteria suspended in media. It also eliminates urea, creatine, uric acid, electrolytes, organic acids, hormones, glucose, amino acids, vitamins, bile salts, drugs, and toxins.

The retentate obtained after the filtration process may contain various components from the original urine sample, such as bacteria, as well as other particles and cellular debris. These can include crystals (e.g., uric acid, calcium oxalate, or phosphate crystals), blood cells (red and white blood cells), epithelial cells from the urinary tract, mucus, and any other insoluble material or contaminants that may be present in the urine. To account for these components during the subsequent analysis or processing steps to ensure accurate results and interpretation, a HisPur Cobalt Chrome column may be optionally integrated into the cartridge to eliminate such contaminants.

Introduction of Antibiotics: After the sample (e.g., the retentate containing bacteria) is processed, it is directed towards a mixing chamber containing the antibiotic-loaded matrix. The matrix is preferably made from a biocompatible material such as hydrogel. This matrix serves as a reservoir for the antibiotic, allowing it to be evenly distributed and facilitating effective mixing with the sample. The sample flows through or around the matrix, allowing it to mix with the antibiotic. Passive or active mixing structures may optionally be used to enhance mixing. Following mixing, the sample-antibiotic mixture can be directed to an incubation chamber where it is incubated for 35 minutes.

According to a preferred embodiment, the antibiotic-loaded matrix is made by thoroughly mixing an antibiotic powder or pellet with the matrix material to ensure a uniform distribution. The mixture of antibiotic and matrix material is then shaped into a pellet, and the solidified antibiotic-loaded matrix is placed into the disposable cartridge. The cartridge is designed to protect the antibiotic and maintain its sterility until it's ready for use. The cartridge may also be designed to facilitate easy handling and application of the antibiotic-loaded matrix during a medical procedure.

The device preferably features eight channels designed for simultaneous testing and analysis, streamlining the process and enhancing efficiency. According to a preferred embodiment, the channels may be configured as follows:
1. Patient's sample: The primary channel containing the patient's urine sample for confirmation of UTI.
2. Negative control: A derived control from the patient's sample to establish a baseline for comparison with antibiotic-treated samples.
3. Trimethoprim (4 μg/mL): A channel containing the patient's sample mixed with Trimethoprim at a concentration of 4 μg/mL to evaluate its efficacy.
4. Fosfomycin (128 μg/mL): A channel with the patient's sample combined with Fosfomycin at a concentration of 128 μg/mL to assess its effectiveness.
5. Cephtriaxone (4 μg/mL): A channel containing the patient's sample mixed with Cephtriaxone at a concentration of 4 μg/mL to test its efficiency.
6. Cephalexin (32 μg/mL): A channel with the patient's sample combined with Cephalexin at a concentration of 32 μg/mL for effectiveness evaluation.
7. Nitrofurantoin (128 μg/mL): A channel containing the patient's sample mixed with Nitrofurantoin at a concentration of 128 μg/mL to determine its potency.
8. QC channel: A quality control channel where Mueller-Hinton Broth (MHB) is tested for contamination, ensuring the accuracy and reliability of the assay results.

Introduction of Reagents: Following mixing of the sample with the antibiotic-loaded matrix, the sample is then further processed by mixing it with an indicator dye (preferably alamarBlue), an electron mediator (preferably Methyl viologen dichloride hydrate), and sodium salicylate to shrink the polysaccharide shell in bacteria that have them. The reagents are stored in fluidic blisters that can be released using a linear actuator.

### Benefit of These Reagents

Sodium Salicylate: This is important for improving the assay's sensitivity and accuracy in certain cases, particularly when dealing with bacteria that have a thick polysaccharide shell or capsule. Polysaccharide shells or capsules in bacteria can act as a barrier, impeding the diffusion of alamarBlue dye and other assay components into the bacterial cells. Consequently, the presence of a thick polysaccharide shell may lead to underestimation of the bacteria's metabolic activity and, in turn, their antibiotic susceptibility.

Methyl Viologen Dichloride Hydrate: Affecting NADH production: Methyl viologen increases NADH production of the bacteria and therefore improves the sensitivity of the device.

After the reagents, including the alamarBlue dye, electron mediator, and sodium salicylate are mixed with the sample, it is directed to the analysis chamber. The analysis chamber is designed to be closed to the atmosphere to increase the sensitivity of the device. The analysis chamber is the part of the cartridge that fits inside the analyzer device.

Analyzer Design: A single-excitation, single-emission kinetics fluorometer is configured to removably receive the fluidic cartridge. It employs a 4-mW, 520-nm, solid-state laser to excite the alamarBlue-containing sample. The dye's active component, resazurin, is reduced to resorufin, which emits light at around 590 nm when excited at 520 nm. The emitted light is detected using a photodiode equipped with a bandpass filter, which selectively transmits the emission wavelength of interest (approximately 590 nm) while blocking other wavelengths. This ensures accurate and sensitive measurements by reducing background noise and interference. The simple yet effective design of the single-excitation, single-emission kinetics fluorometer allows for real-time monitoring of bacterial growth and antibiotic susceptibility in various applications.

Bacterial concentration determination: The present invention uses the alamarBlue assay to measure the fluorescence change over time, which correlates with bacterial concentration in urine. An algorithm involves measuring the initial fluorescence, continuously monitoring fluorescence at regular intervals, calculating the change in fluorescence, and determining the rate of change. By comparing the rate of fluorescence change to a pre-established calibration curve, the method and device of the invention can estimate the range of bacterial concentration in the sample, streamlining the diagnosis and treatment of urinary tract infections.

Antibiotic Resistance Determination: The multi-channel system of the invention enables simultaneous testing of different antibiotics. By monitoring the fluorescence change in each channel and comparing it to pre-determined cutoff values, the system can identify which antibiotics are effectively inhibiting bacterial growth.

The alamarBlue assay relies on the reduction of resazurin to resorufin, a process that is dependent on bacterial metabolic activity. As the concentration of bacteria in the urine increases, so does the rate of resazurin reduction, leading to a more rapid increase in fluorescence. The inventive algorithm for determining the range of bacterial concentration in urine based on the fluorescence change over time is as follows:
1. Measure the initial fluorescence (F0) of the sample.
2. Start the timer and continuously measure the fluorescence at regular intervals (e.g., every minute) for a predetermined period (e.g., 15 minutes).
3. Calculate the change in fluorescence (AF) at each time interval by subtracting F0 from the current fluorescence value (Ft).
4. Plot the AF values over time to generate a fluorescence curve.
5. Determine the slope of the curve (rate of fluorescence change) by calculating the difference in AF between two consecutive time points and dividing by the time interval.
6. Compare the slope of the curve to a pre-established calibration curve, which correlates the rate of fluorescence change to known bacterial concentrations.
7. Identify the range of bacterial concentration in the urine sample based on the position of the slope on the calibration curve.

It will be appreciated by those skilled in the art that changes could be made to the preferred embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A method for rapid diagnosis and antibiotic susceptibility testing of urinary tract infections, comprising the steps of:
a. collecting a urine sample from a patient,
b. transferring the urine sample into a fluidic cartridge,
c. performing a buffer exchange to isolate and concentrate bacteria present in the urine sample,
d. introducing a panel of antibiotics to the concentrated bacterial suspension,
e. incubating the bacteria-antibiotic mixtures for a predetermined period,
f. introducing a reagent to assess bacterial metabolic activity,
g. determining bacterial concentration in the original urine sample based on fluorescence readings, and
h. determining antibiotic susceptibility by comparing fluorescence intensities with predetermined threshold values.

2. The method of claim 1, wherein the buffer exchange process involves passing the urine sample through a series of filters and buffer solutions to separate bacteria from other cellular debris and contaminants.

3. The method of claim 1, wherein the panel of antibiotics includes a range of antibiotics commonly used to treat urinary tract infections.

4. The method of claim 1, wherein the reagent for assessing bacterial metabolic activity is alamarBlue.

5. A fluidic cartridge for use in the method of claim 1, comprising:
a. a sample input for receiving the urine sample,
b. a buffer exchange module for isolating and concentrating bacteria present in the urine sample,
c. a plurality of microchambers for accommodating bacteria-antibiotic mixtures, and
d. a reagent introduction module for introducing the reagent to assess bacterial metabolic activity.

6. An analyzer for use in the method of claim 1, comprising:
a. a solid-state laser for excitation,
b. a photodiode with a bandpass filter for detecting emitted light, and
c. a processor for calculating bacterial concentration in the urine sample and determining antibiotic susceptibility based on fluorescence readings.

7. The method of claim 1, wherein filtration, incubation and analysis takes place within a total duration of less than 60 minutes.
